(19) United States
(12) Patent Application Publication
(10) Pub. No.: US 2026/0338736 A1
(43) Pub. Date: Oct. 29, 2026

(54) ACTIVE SENSOR DEVICES AND METHODS FOR DETECTING FERROMAGNETIC METAL DEBRIS

(71) Applicant: KASPERAERO LLC, Kent, OH (US)

(72) Inventor: Benjamin KASPER, Kent, OH (US)

(21) Appl. No.: 19/567,756

(22) Filed: Mar. 16, 2026

Related U.S. Application Data
(60) Provisional application No. 63/795,280, filed on Apr. 26, 2025.

(51) Int. Cl. G01N 27/74; G01N 33/28; G01V 3/08

(52) U.S. Cl. CPC G01N 27/74; G01N 33/2858; G01V 3/08

(57) ABSTRACT

Active sensor devices and methods for detection of ferromagnetic metal debris in a fluid. The sensor devices include a magnetic field sensor preferably arranged in a null zone or area of the sensor device wherein a magnetic field of a magnet of the device is not able to be detected by the sensor. The sensor devices detect ferromagnetic metal debris by measuring changes in the magnetic field produced by the magnet via the magnetic field sensor. The sensor devices are particularly useful for detection of wear particles in a lubricant or fuel.

### FIELD OF THE INVENTION

The present invention relates to active sensor devices and methods for detection of ferromagnetic metal debris in a fluid. The sensor devices include a magnetic field sensor preferably arranged in a null zone or area of the sensor device wherein a magnetic field of a magnet of the device is not able to be detected by the sensor. The sensor devices detect ferromagnetic metal debris by measuring changes in the magnetic field produced by the magnet via the magnetic field sensor. The sensor devices are particularly useful for detection of wear particles in a lubricant or fuel.

### BACKGROUND OF THE INVENTION.

Many different devices have been proposed in the art for sensing particles, in particular ferromagnetic particles in the form of unwanted debris in fluids such as lubricants and fuels.

Machine parts, such as aircraft engines and gear boxes in which components move relative to each other, are often lubricated with a lubricant oil to reduce wear. Many types of fuel systems are recirculatory in nature and rely on the lubricity of the fuel for the proper operation of the pump. However, over time, small wear particles with sizes in the range of 1-10 microns (Lm) are generated. When abnormal wear begins, larger particles, in the range of 10 to 50 microns are generated. The particle population and size of the particles tends to increase over time until eventually, a machine failure can result.

Many industries such as automotive, aerospace, and industrial have contented themselves with magnetic drain plugs which collect the ferrous debris for visual inspection. Some industries such as aerospace will take these collected magnetic particles and analyze their composition to determine which engine components are wearing. Aerospace has also made these magnetic drain plugs convenient to access through the use of quick release mechanisms and self-closing valves which allow the collected debris to be analyzed without draining the lubrication system. These magnetic drain plugs, so called magnetic chip collectors are popular because of their low cost, high reliability and passive nature.

The aerospace market has also come to adopt passive magnetic chip detectors. These passive chip detectors consist of permanent magnets which serve to attract the ferrous debris and electrodes which are bridged when covered with ferrous debris. They rely on the chips they are collecting to be conductive to complete the circuit.

The two main styles of chip detectors are the prong style, see FIG. 5, and disc style, see FIG. 6. In the prong style chip detectors, the magnets serve to attract the debris and as the detecting electrodes. In the disc style, the magnet is not a part of the electronic circuit.

These sensors have wide adoption because of they have no internal circuitry, no programing, and no calibration which makes them highly reliable and easy to qualify to aerospace standards. Another benefit of these sensors is that the debris they collect can be easily visually analyzed or sampled and sent for lab analysis. However, these sensors cannot sense very fine wear particles in the range of 1-10 microns (Lm) because they rely on the ferrous debris to be conductive. Extremely fine ferrous dust is not very conductive, especially when coated in oil. Another drawback to these sensors is the possibility that the debris that is collected is not bridging the electrodes, see FIG. 7.

In this way, these sensors rely on probability and randomness and thus are not perfectly reliable.

Relatively fine magnetic dust can be detected by many so-called active sensors. These systems involve microcontrollers, programming, calibration and power electronics to detect ferrous debris in lubrication systems. These methods include microfluidic devices measuring magnetic flux field distortion using a sensing electromagnetic coil, tribological approaches, optical approaches such as scattering counters, capacitive measurement methods, Ferrography, acoustic methods, and inductive measurement sensors. While these active sensors are accurate and able to sense small quantities of ferrous material, they are relatively costly, not compact, and not generally robust enough for harsh environments such as aerospace. Additionally, some of these methods do not capture the ferrous debris in a manner that allows for easy visual analysis of the debris or capture for lab analysis.

Various proposals have been presented in patent literature for active sensing of ferromagnetic metal debris in lubricating or other fluids.

EP0116468 relates to a magnetic particle collector for magnetically attracting particles from a lubricating or other fluid and has a permanent magnet in a housing of non-magnetic material which is retractable from an active position where it attracts particles to the outside of the housing to an inactive position which permits particles attracted to the outside of the housing to be flushed away by the fluid flow. The collector has a means such as a Hall effect device or a magnetic field sensitive resistor for sensing the amount of particles attracted to the housing by responding to the distortion of the magnetic field due to the accumulation of particles.

U.S. Pat. No. 12,158,048 relates to a wellbore magnet tool including a plurality of magnets positioned about an outer circumference of the wellbore magnet tool; a plurality of sensors, including: a first sensor proximal one or more of the plurality of magnets; and a second sensor positioned downhole from the first sensor, and an electronics board. The first sensor and the second sensor are electronically connected with the electronics board, and the first sensor and the second sensor detect changes in a magnetic field proximate thereto, which changes result from debris collection by the wellbore magnet tool.

U.S. Pat. No. 12,216,084 relates to methods and systems for an oil sensor. In one example, the oil sensor is a system for metallic debris detection, comprising a detection circuit including a first inductor and a second inductor, the second inductor shielded from the external environment, wherein the detection circuit generates an output based on a difference between a first voltage of the first inductor and a second voltage of the second inductor, where the difference indicates a presence of metallic debris within oil.

U.S. Pat. No. 8,522,604 relates to an apparatus and a method for detection of wear particles in a lubricant. The apparatus includes a microfluidic device including a microchannel sized for a lubricant containing wear particles to pass therethrough and first and second electrodes extending into the microchannel. A detection system is coupled with the electrodes for detection of wear particles passing through the microchannel, based on a change in capacitance of the electrodes.

Even in view of the above, the art still needs reliable active sensor devices for detecting ferromagnetic metal debris in a fluid.

There remains a need for a detection method which permits a sensor to be designed that is robust, low-cost, compact, performs in-situ testing of lubricants or fuels that allows a rapid response when wear particles accumulate, is repeatable, allows for visual inspection of the collected debris, and which is applicable to wear particles that are ferromagnetic but not electrically conductive.

### SUMMARY OF THE INVENTION

As a solution, the present invention provides a sensor device and a method for detection of wear particles in a fuel or lubricant. The sensor device includes an inventive magnetic arrangement and precise positioning of a magnetic field sensor with respect to the magnet that allows detection of wear particles that are collected by the magnet, based on an altered magnetic field in the presence of ferromagnetic metal debris.

An important advantage of the sensor devices of the invention is the fact that the magnetic field sensor is placed at the null zone where there is no magnetic field present. The null zone is a place where the magnetic field is zero in all axes. This gives the debris sensor device unparalleled stability throughout the temperature range, simplicity that other sensor devices do not have, and the ability to use as strong as a magnet as can be desired.

The sensor devices are robust, low-cost, compact, perform in-situ testing of lubricants or fuels that allows a rapid response when wear particles accumulate, are repeatable, allow for visual inspection of the collected debris, and which are applicable to wear particles that are ferromagnetic but not electrically conductive.

Importantly, the sensor devices do not have electrodes exposed to the fluid environment.

In accordance with one aspect or embodiment of the present invention, an apparatus for detection of wear particles in a fluid such as, but not limited to, lubricant or fuel, is disclosed and includes one or more strong permanent magnets and a magnetic field sensor. The magnets are sized large enough to attract and retain the wear particles as they pass by the sensor. The magnetic field sensor is sensitive enough to sense a small amount of wear particles. To make a sensor that is sensitive enough to detect the small ferrous particles, with a magnet large enough to attract these particles, the magnets must be arranged relative to the magnetic field sensor in such a way that the magnetic field sensor does not become saturated.

As disclosed herein there are several configurations of magnets and magnetic field sensors that allow highly sensitive magnetic field sensors to operate without becoming saturated while still being able to detect a change in magnetic field that indicates the presence of ferrous debris.

In one aspect or embodiment a sensor device for detecting ferromagnetic metal debris in a fluid is disclosed, comprising a housing having a sensor head unit adapted to be exposed to a fluid potentially containing ferromagnetic metal debris, wherein the housing comprises a non-ferromagnetic material; a magnet disposed in the housing, wherein the magnet generates a first magnetic field, a magnetic field sensor disposed in the housing, wherein the magnetic field sensor is positioned at a location where the first magnetic field is not able to be detected by the magnetic field sensor, and wherein during use the magnet attracts ferromagnetic metal debris that becomes attached to the housing adjacent to the magnetic whereby the first magnetic field shifts and becomes a second magnetic field, and wherein the second magnetic field is detected by the magnetic field sensor.

In a further aspect or embodiment, the magnet and magnetic field sensor are disposed in the head unit.

In an additional aspect or embodiment, the magnetic field sensor is one or more of a Hall effect sensor, an anisotropic magneto resistive sensor, a giant anisotropic magnetic resistive sensor and an anisotropic tunneling magnetoresistive sensor.

In yet a further aspect or embodiment, the head unit has a front end, and wherein the magnetic field sensor is located closer to the front end of the head unit as compared to the magnet.

Still another aspect or embodiment, the magnet is one or more of a ring magnet, a plurality of one or more of rod and bar magnets arranged in the form of the ring, two rod magnets or two bar magnets.

In a further aspect or embodiment, the sensor head unit is substantially cylindrical in shape.

In an additional aspect or embodiment, the magnetic field sensor is located closer to a radial center of the head unit as compared to the magnet.

In yet a further aspect or embodiment, the housing includes a base connected to the sensor head unit, wherein a printed circuit board is located within the base and has an arm that extends upwardly into the sensor head unit, and wherein the magnetic field sensor is connected to the arm of the printed circuit board.

Still another aspect or embodiment, the wiring is connected to the circuit board for controlling the magnetic sensor, and wherein the wiring extends outwardly from the base.

In a further aspect or embodiment, a first pole of the magnet is disposed closer to a front end of the magnetic field sensor head unit as compared to a second opposite pole of the magnet.

In an additional aspect or embodiment, a method for detecting ferromagnetic metal debris in a fluid is disclosed comprising the steps of: obtaining the sensor device; connecting the sensor device to an apparatus whereby the sensor head unit comes into contact with a fluid potentially containing ferromagnetic metal debris; evaluating the fluid for ferromagnetic debris; and sending an alert to an operator when a threshold debris level has been sensed.

In still another aspect or embodiment, the step of connecting the sensor device to the apparatus includes placing the sensor device in the apparatus at a location where fluid flows past the sensor.

In yet a further aspect or embodiment, the step of connecting the sensor device to the apparatus includes incorporating the sensor into a reservoir of the assembly.

In an additional aspect or embodiment, a method for forming the sensor device is disclosed comprising the steps of: obtaining the magnet that generates the first magnetic field; determining a location where the first magnetic field is not able to be detected by the magnetic field sensor; positioning the magnetic field sensor at the location; and incorporating the magnet and magnetic field sensor in the housing.

### BRIEF DESCRIPTION OF THE DRAWINGS

The invention will be better understood and other features and advantages will become apparent by reading the detailed description of the invention, taken together with the drawings, wherein:

FIG. 1 is a cross-sectional view of one embodiment of a sensor device of the present invention for detecting ferromagnetic metal debris in a fluid;

FIG. 2 illustrates a magnetic field sensor positioned in a null zone, free or substantially free of a magnetic field produced by a magnetic arrangement utilized in the present invention, with a cross-sectional view of a ring magnet being shown;

FIG. 3 illustrates a shift in the magnetic field produced by a magnet utilized in the device of the present invention induced by debris attracted to the magnet which alters the magnetic field so that it is sensed by the magnetic field sensor;

FIG. 4 illustrates one embodiment of a block diagram of an electrical circuit utilized in the sensor device of the present invention;

FIG. 5 illustrates one embodiment of a prior art prong-style debris detector;

FIG. 6 illustrates one embodiment of a prior art disc-style debris detector;

FIG. 7 illustrates a prior art sensor wherein debris does not bridge the electrodes;

FIG. 8 depicts a graph of magnetic flux density along the centerline of a ring magnet;

FIG. 9 depicts the formula utilized to create the graph shown in FIG. 8;

FIG. 10 depicts the magnetic field surrounding a rod magnet using an magnetic field graph;

FIG. 11 depicts the horizontal component of the magnetic field surrounding a rod magnet using a vector field graph;

FIG. 12 depicts a graph illustrating the line where the horizontal component of the magnetic field changes polarity;

FIG. 13 depicts two rod magnets arranged to create a null zone;

FIG. 14 depicts the magnetic flux density along the centerline of the two parallel rod magnets;

FIG. 15 depicts the formula used to create the FIGS. 8 and 10 through 12;

FIG. 16 depicts a graph of magnetic flux density along the centerline of a ring magnet of the same geometry at various magnet strengths;

FIG. 17 depicts a graph of magnetic flux density along the centerline of a rod magnet arrangement of the same geometry at various magnet strengths; and

FIG. 18 depicts how a small amount of ferromagnetic debris influences a readout of a sensor.

### DETAILED DESCRIPTION OF THE INVENTION

This description of preferred embodiments is to be read in connection with the accompanying drawings, which are part of the entire written description of this invention. In the description, corresponding reference numbers are used throughout to identify the same or functionally similar elements. Relative terms such as “horizontal,” “vertical,” “up,” “upper”, “down,” “lower”, “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and are not intended to require a particular orientation unless specifically stated as such. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The term “operatively connected” is such an attachment, coupling or connection that allows the pertinent structures to operate as intended by virtue of that relationship.

The sensor devices of the present invention are particularly useful for detecting ferromagnetic debris in a fluid that can flow past a sensor head unit of the sensor device. The sensor devices are well suited for placement in reservoirs or sumps of an assembly or construction where the magnet can attract ferromagnetic metal debris over time. It is to be understood that while the attention of the sensor devices is focused on ferromagnetic metal debris, applications of the sensor devices also include relatively larger ferrous objects or magnets, among others.

Turning now to the drawings, wherein like reference numbers represent like or the same parts throughout the several views, FIG. 1 illustrates a cross-section of one embodiment of a sensor device 10 of the present invention. Sensor device 10 includes a housing 20 which protects the internal components including a magnetic field sensor 30, magnet 40 and circuitry 50.

### Housing

Housing 20 services as a durable enclosure for the active components of the sensor device 10. In some embodiments, in order to avoid interference with the magnet, the housing comprises non-ferromagnetic material. However, ferromagnetic materials may be used at locations of the sensor housing 20 which do not interfere with the magnetic field generated by the magnet. Housing 20 may be formed of any suitable material that can withstand the environment in which the sensor device 10 is utilized. For example, suitable materials include, but are not limited to stainless steel, aluminum alloys, polyimides, and polyaryletherketones.

Housing 20 includes a sensor head unit 22 operatively connected to a base 24. Sensor head unit 22 is adapted to be exposed to a fluid potentially containing ferromagnetic metal debris. In one embodiment, the sensor head unit is inserted into an orifice of a mechanical assembly containing a fluid to be monitored for ferromagnetic debris such that at least a front end of the sensor is in direct contact with the fluid. This may be accomplished by operatively connecting the sensor device 10 to the equipment utilizing a quick release mechanism 25, which allows for relative ease of insertion and removal of the sensor device from the assembly to be monitored. One preferred method of connection of the sensor device and assembly is a bayonet mount. Other connections known in the art can be utilized alternatively.

The sensor housing 20 may include features such as a ring groove 26 which accepts an O-ring that aids in sealing sensor housing 20 the desired assembly and prevents fluid leaks between the same.

In the embodiment illustrated in FIG. 1, the housing 20 includes a rear end having a cord grip with strain relief for wiring 60 that provides power to the sensor device 10.

In one embodiment, the front of the housing 20 sensor head unit 22 includes a cap 23 formed from a material that is different from other material utilized in the sensor head unit 22. In some embodiments, the material is inert and therefore does not have any affect on the magnetic field generated by the magnet. In such cases the cap 23 may be formed from a polymer such as epoxy. In some embodiments UV-curable epoxy is utilized. In other embodiments, the cap 23 material may aid in collecting ferromagnetic metal debris on the sensor head unit. In such cases, the cap 23 may be formed from a suitable metal such as, but not limited to stainless steel and an aluminum alloy. In additional embodiments, the housing 20 may include features that make it easy to grasp, which facilitates connection and disconnection from an assembly. The housing may also be provided with alignment-indicating grooves or other features to ensure proper orientation in relation to an assembly.

### Magnet

The sensor device 20 of the present invention include one or more magnets 40 arranged in the sensor head unit 22.

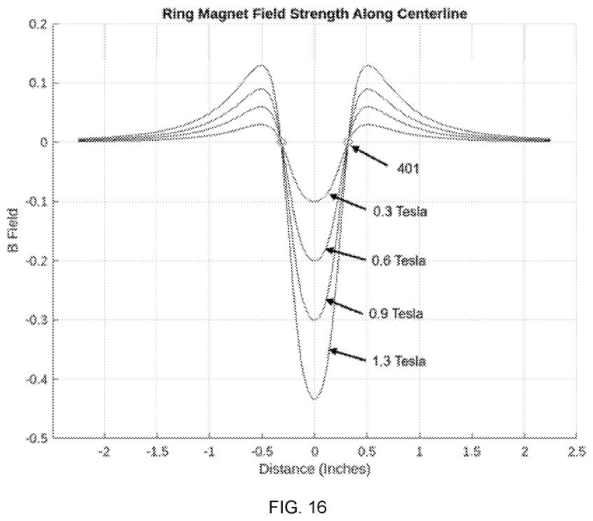

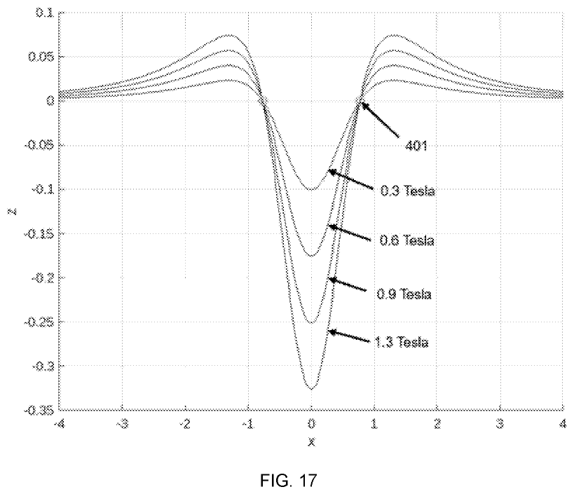

The preferred magnets utilized in the present invention are designed to operate across the wide temperature range the sensor device may encounter during use without appreciably changing in magnetic field strength across the operating temperature range. Unlike prior art, wherein the output of the sensor must be altered using a temperature to sensitivity map, this sensor is inherently temperature stable. The location of the null zone does not appreciably move with temperature, see FIG. 16 and FIG. 17). This minimizes variation across a wide temperature range.

The magnets utilized in the present invention are preferably permanent magnets. Permanent magnets retain their magnetic properties and generate a magnetic field without the use of electricity. Suitable permanent magnets which can be utilized in the present invention include Alnico magnets, ceramic magnets, neodymium magnets and samarium magnets. Alnico magnets comprise aluminum, nickel and cobalt with small amounts of other elements which are added to enhance the properties of the magnet. Ceramic magnets are generally a composite of iron oxide and barium or strontium carbonate. Neodymium magnets generally comprise neodymium, iron and boron. Samarium magnets generally comprise samarium and cobalt. Neodymium magnets are preferred in some embodiments of the present invention.

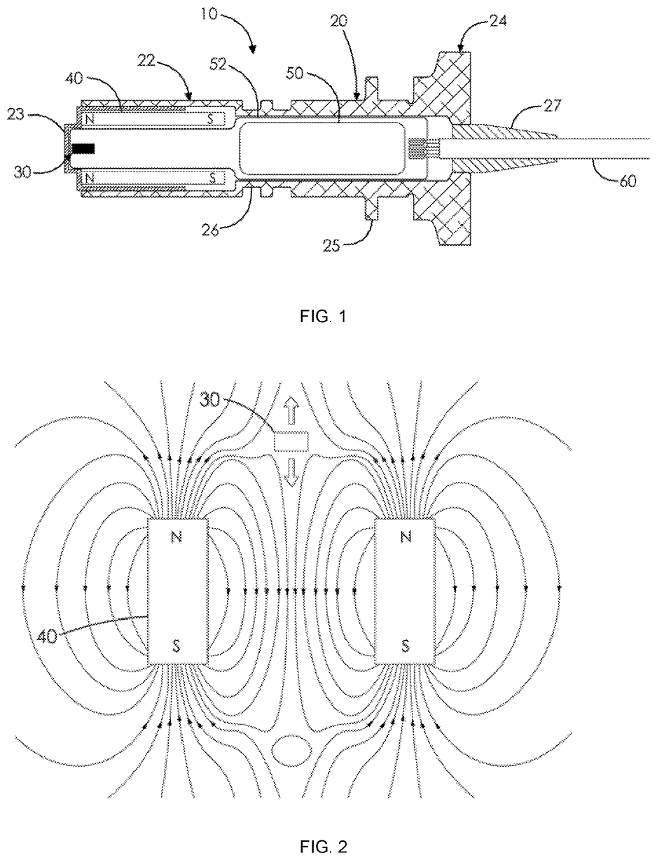

Another important feature of the present invention is the structural arrangement of the magnets in the sensor device. Three primary types of structural arrangements can utilized. Ring magnets are utilized in one embodiment and form a desirable magnetic field including a null zone or area where no magnetic field is present, for example see FIG. 2. In other embodiments, multiple rod or bar magnets can be utilized in a sensor device arranged in the form of a ring. In still other embodiments, two rod or bar magnets can be used, wherein the individual magnets are spaced from each other a predetermined distance in order to generate an area of the sensor device where no magnetic field is present and at which the magnetic field sensor can be placed.

The magnetic field of a ring magnet or multiple magnets arranged in the form of a ring changes polarity suddenly at a null zone. In this area, the magnetic field is zero, but is surrounded by stronger magnetic fields on either side of opposite polarity. By placing the magnetic field sensor in a null zone, it will read a magnetic field of zero. When ferromagnetic material 60 is attracted to the ring magnet and attached to an outer surface of the sensor device, in particular to the sensor head unit, the debris affects and changes the magnetic field and essentially relocates the null zone. The modified magnetic field then extends to the location of the sensor device where the magnetic field sensor is located and is detected thereby, for example as illustrated in FIG. 3.

This structural arrangement and sensing method provides a desirable advantage across temperature extremes because while the magnet strength varies through the temperature range, the null zone does not move. In this way, the magnetic field sensor can be placed outside of a first magnetic field without becoming saturated and detect the presence of ferromagnetic debris when the debris induces a second magnetic field or otherwise alters and extends the original, first magnetic field.

In some embodiments, order to affix the magnets in the head unit the magnets are placed in a plastic carrier and set in place, preferably with an adhesive or glue such as epoxy. In some embodiments UV-curable epoxy is utilized.

If the sensor device is to operate over a very broad temperature range, the sensor and magnets must be designed so that their positions are stable across the temperature spectrum. Any polymeric or adhesive components utilized in the sensor desirably have low Coefficients of Thermal Expansion (CTE).

The magnets utilized in the present invention preferably have uniform magnetization. Axially magnetized ring and rod magnets are preferred.

### Sensors

The magnetic field sensors utilized in the present invention are a specialized subset of a proximity sensor. Their design allows them to be extremely repeatable because they are not affected by the strength of the magnet. They are also repeatable because they do not need to take measurements at the extremes of the integrated circuit sensor's range. The sensors of the present invention are very sensitive to debris and if the sensor is utilized as a proximity sensor, the sensor needs to be shielded from debris. Both analog sensors and digital sensors can be utilized in the present invention.

There are several types of sensors suitable for use in the present invention. The sensors are preferably integrated circuit sensors. Suitable sensors that can be used in the present invention include, but are not limited to, Hall effect sensor, an anisotropic magneto resistive sensor, a giant anisotropic magnetic resistive sensor and an anisotropic tunneling magnetoresistive sensor. The magnetic field sensors utilized in the present invention should have an operating temperature range greater than the expected temperature range encountered by the debris sensor devices. Sensors with high sensitivity are desirable. Low power consumption is also preferred. Suitable sensors are commercially available from sources such as Allegro Microsystems of Manchester, NH; Honeywell of Charlotte, NC; NVE Corporation of Eden Prairie, MN; Coto Technology of East Greenwich, RI as RedRock® Sensors and Texas Instruments of Dallas, TX.

### Circuitry

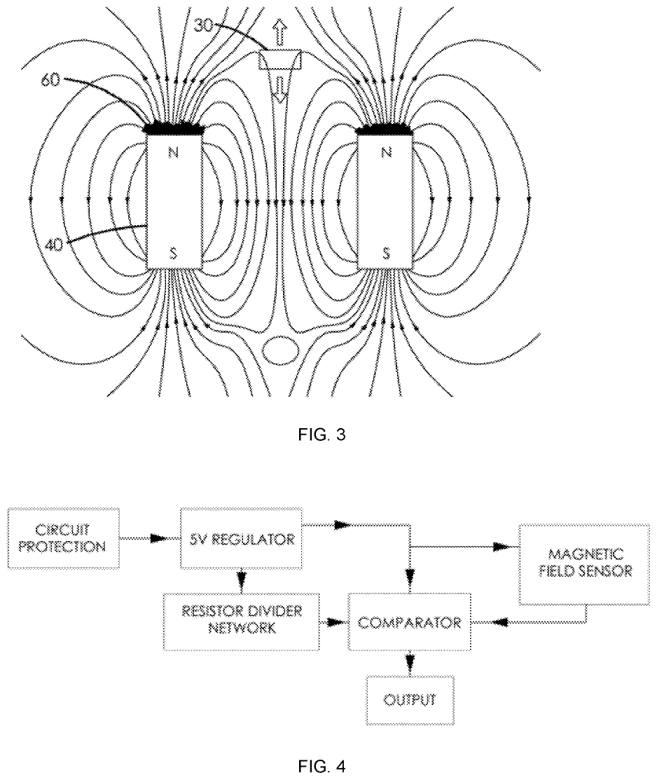

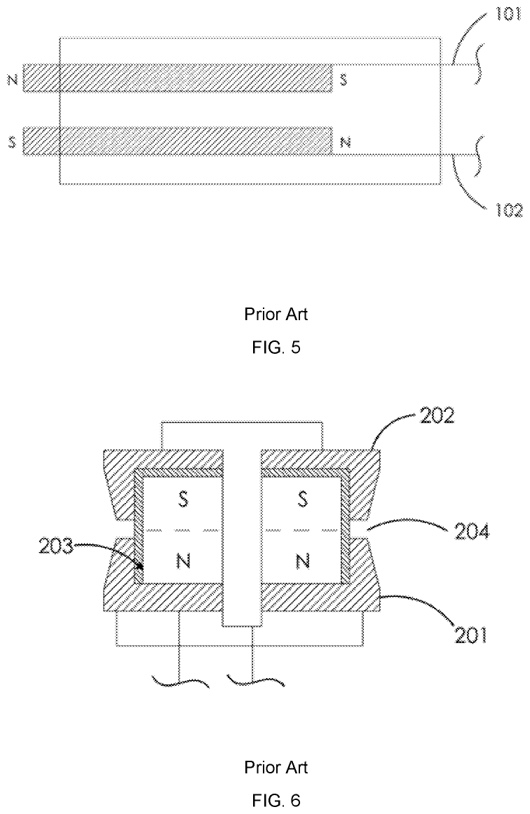

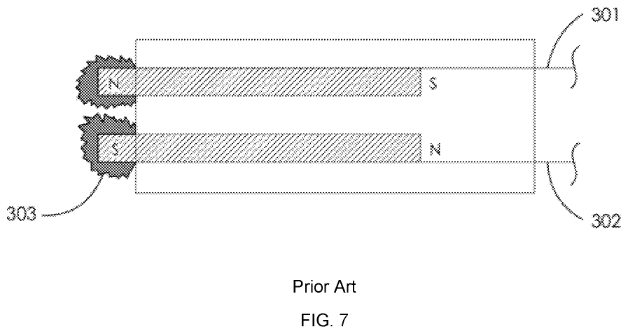

The sensor devices 10, 110 of the present invention include circuitry 50 including a printed circuit board 52. A block diagram for one preferred embodiment for circuitry 50 is illustrated in FIG. 4. Circuit Protection.

Circuit protection includes a silicon reverse polarity protection diode to protect against incorrect wiring. It also a transient-voltage-suppression TVS diode to protect against high voltage transients (high voltage short duration spikes). It also includes a low value resistor and low value capacitor to protect the TVS diode and silicon diode.

### 5V Regulator

A regulator may or may not be necessary depending on the voltage range of the sensor.

### Resistor Divider Network

The resistor divider network is a series of bog-standard thin film resistors used to create reference voltages that are needed for comparison.

### Comparator

The comparator compares the voltage output of the magnetic field sensor to the voltages of the resistor divider network. It is important that the comparator has a low DC offset voltage.

### Output

The output block uses an N-channel mosfet to create a safe output signal.

### Locating the Magnetic Null Zone

The sensor devices of the invention rely on precise placement within the null zone of a magnetic field, which is the spatial region where the net magnetic flux density is approximately zero. Determining this null zone is dependent upon the geometry and configuration of the selected magnets.

For ring magnets, the null zone can be calculated using a closed-form equation based on the magnet's dimensions and material properties. In contrast, for rod or bar magnet configurations, the null zone must be identified through iterative or numerical methods, as no simple analytical expression exists. In both cases, the location can also be determined empirically by incremental adjustments of a magnetic field sensor until a zero field reading is observed.

### Flux Density along Centerline of a Ring Magnet

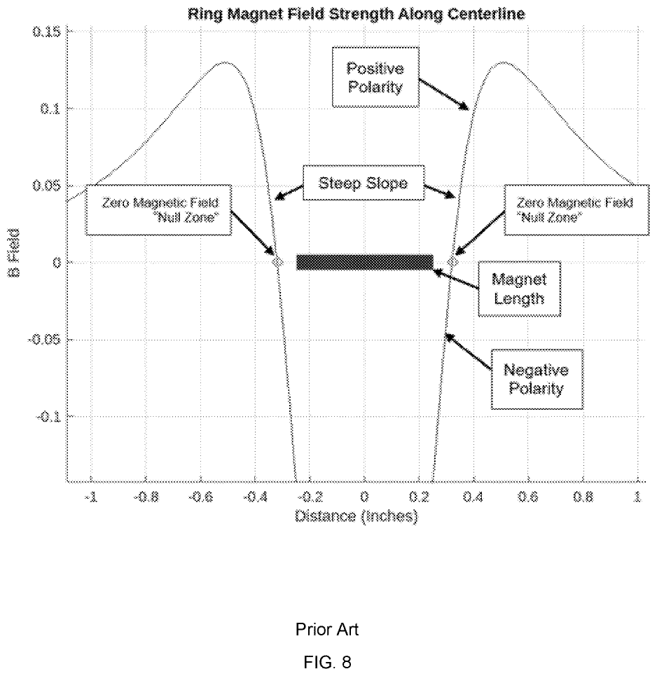

FIG. 8 depicts a graph of magnetic flux density along the centerline of a ring magnet. There are strong opposite polarity magnetic fields on either size of the null zone. The null zone extends just past the face of the ring magnet. The distance from the null zone to the face of the ring magnet is a function of the magnet geometry.

At the null zones, the magnetic field is zero in the X, Y, and Z directions despite being very close to a ring magnet.

FIG. 8 depicts the magnetic field along the centerline of a ring magnet of the following dimensions:
Length (D): 0.50″
Outer Diameter (Ra): 0.50″
Inner Diameter (Ri): 0.20″
Remanence (Br): 1.3 Teslas.

The region where the magnetic field is negligibly small creates a zone that the magnetic field sensor can be placed in.

### Magnetic Flux along the Centerline of Ring Magnet

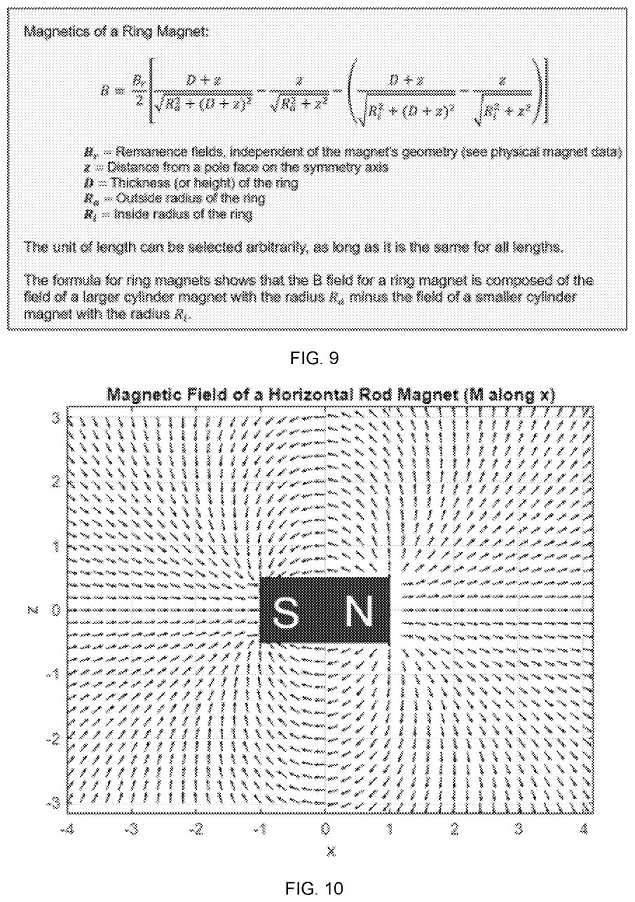

FIG. 9 depicts the formula needed to create the graph as shown in FIG. 8. A null zone occurs at the “zeroes” of this equation, when B=0. This is best determined using numerical methods.

### Magnetic Field of a Horizontal Rod Magnet

FIG. 10 depicts the magnetic field surrounding a rod magnet using a vector field graph. Arrow lengths are uniform for clarity. They depict field direction but do not depict field strength.

### Horizontal Component of a Magnetic Field of a Rod Magnet

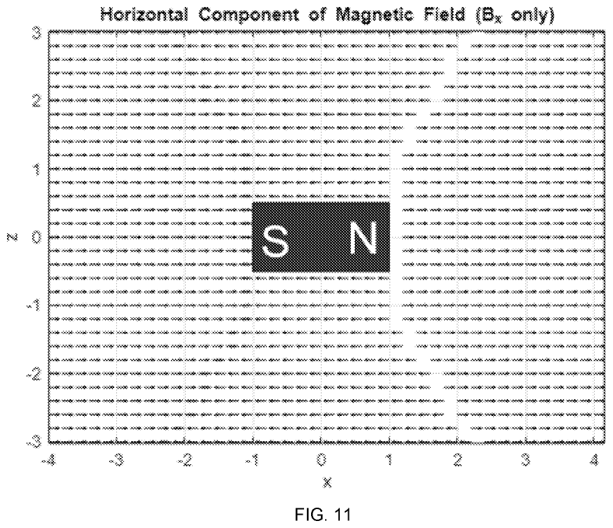

FIG. 11 depicts the horizontal component of the magnetic field surrounding a rod magnet using a vector field graph. This graph represents the same magnet as portrayed in FIG. 10.

These arrows have been oriented based on the polarity of the magnetic field along the x-axis. Arrow lengths are uniform for clarity and do not depict strength.

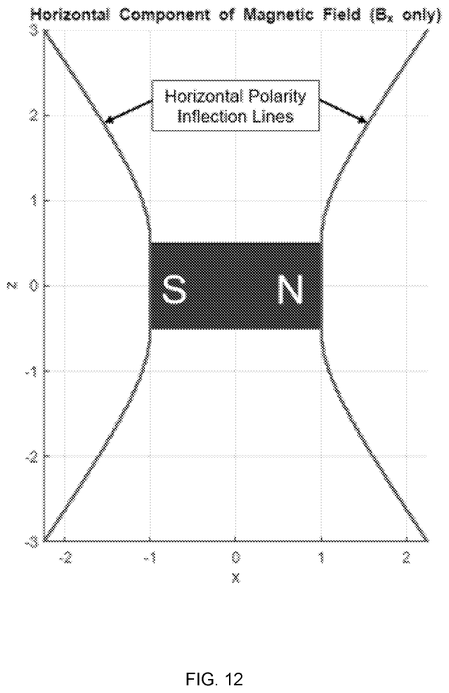

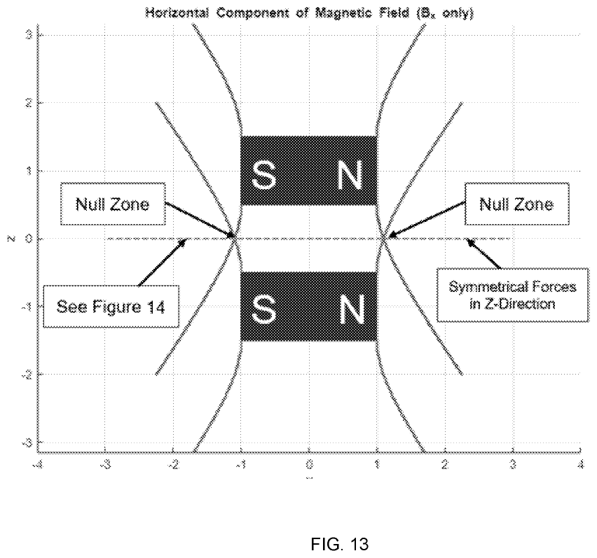

It can be seen that the horizontal component of the magnetic field splits along a line, see FIG. 12. A magnetic sensor that has strictly X-axis sensitivity placed anywhere on this line would read a zero magnetic field and would allow for debris detection. However, magnetic sensors have slight sensitivity to the other axes. Because of this, the sensors will not read zero. Therefore, a single rod magnet cannot be used to make a debris detection sensor.

### Rod Magnet Horizontal Polarity Inflection Lines

FIG. 12 depicts the line where the horizontal component of the magnetic field changes polarity. These lines are referred to as “horizontal polarity inflection lines”.

### Two Rod Magnets Arranged to Create a Null Zone

FIG. 13 depicts the horizontal polarity inflection lines of two rod magnets that are near to each other. Anywhere on these two lines there is no magnetic field in the X direction. Anywhere on the dashed line, the Z-component of the magnetic fields cancel out and there is a net zero magnetic field in the Z-direction. There is no field component in the Y direction. Therefore, at the intersection of these lines there is a null zone. At the null zone, the magnetic field is zero in the X, Y, and Z directions despite being very close to two strong magnets.

On the 2D graph, the null zone is shown as a point. However, because the rod magnets (or bar magnets) are uniform in the Y axis, a line is created that extends into and out of the page along the Y axis.

The region where the magnetic field is negligibly small creates a zone or area that the magnetic field sensor can be placed in.

### Magnetic Field of along the Centerline of a Rod Magnet Arrangement

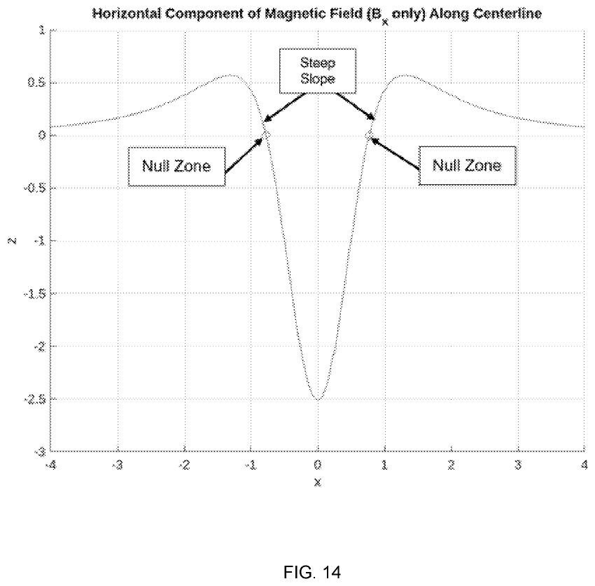

FIG. 14 depicts the magnetic flux density along the centerline of the two parallel rod magnets. This is a graph of the magnetic field strength along the indicating line of FIG. 13.

This graph is comparable to FIG. 8 which shows the magnetic field along the centerline of a ring magnet. Both graphs have steep slopes on either side of the null zone.

### Magnetic Flux for Rod Magnet

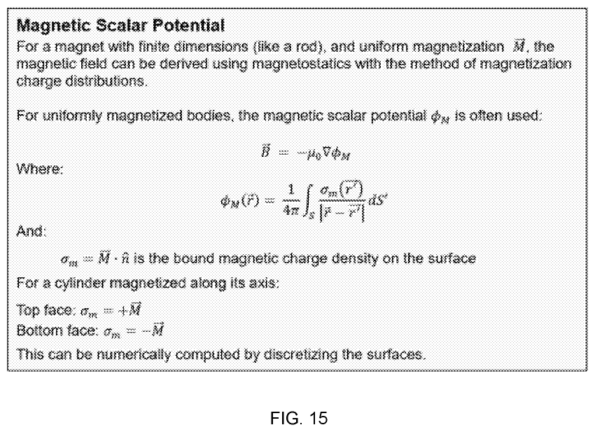

FIG. 15 depicts the formula used to create the FIGS. 8 and 10 through 12.

### Ring Magnet Null Zone Temperature Stability

FIG. 16 depicts a graph of magnetic flux density along the centerline of ring magnet at various magnet strengths. These altered magnet strengths simulate how the strength of a magnet might change with temperature variation. It can be seen that the null zone does not move as magnet strength changes.

### Rod Magnet Null Zone Temperature Stability

FIG. 17 depicts a graph of magnetic flux density along the centerline of a rod magnet arrangement at various magnet strengths. These altered magnet strengths simulate how the strength of a magnet might change with temperature variation. It can be seen that the null zone does not move as magnet strength changes.

### Magnet with Ferrous Debris

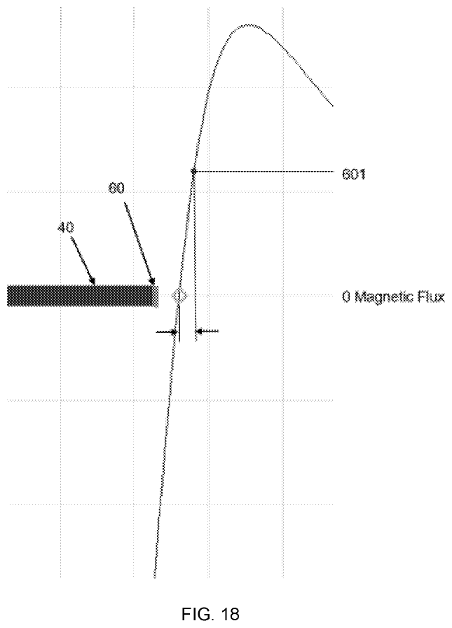

FIG. 18 depicts how a small amount of ferromagnetic debris influences a readout of a sensor. This graph also shows the near linear relationship between debris collected and the change in output.

During device assembly, the magnet or magnets are first affixed into a magnet carrier component. This carrier provides mechanical alignment features to facilitate the proper positioning of the sensor along the magnetic centerline. The sensor (typically integrated on a printed circuit board) is then manually adjusted along this axis until it registers a near-zero magnetic field. At this position, the sensor is secured preferably using and adhesive such as epoxy or potting material, thereby fixing it in place within the null zone. In some embodiments UV-curable epoxy is utilized.

### Assembly and Integration Process

The sensor device is constructed and installed as follows:

One or more magnets are bonded (e.g., using epoxy) into a magnet carrier, which is a component designed to maintain the spatial orientation and stability of the magnets relative to the sensor.

The sensor circuit board, containing a magnetic field sensor and associated electronics (e.g., an LED indicator), is incrementally adjusted within the magnetic field until the sensor output indicates a zero or near-zero reading—signifying alignment with the null zone.

Once aligned, the circuit board is affixed to the carrier preferably using adhesive or potting compound to maintain this precise position.

The assembled sensor and magnet carrier are then enclosed within a protective housing, which may also be filled or capped with epoxy or a metal closure to provide environmental sealing and mechanical protection. In some embodiments UV-curable epoxy is utilized.

The entire sensor unit can then be installed into a larger construction or assembly where ferromagnetic debris monitoring is desired.

### Detection of Ferromagnetic Debris

The core functionality of the device relies on the principle that ferromagnetic debris entering the magnetic field will disturb the balance at the null zone, causing a measurable shift in the magnetic field at the sensor. Because the null zone is located at a steep gradient of the magnetic field, even minute displacements or disturbances (such as from the presence of metallic particles) result in a significant change in sensor output.

FIG. 18 illustrates this behavior, showing how the field strength gradient enables high sensitivity to perturbations caused by nearby ferrous particles. The change in the magnetic environment is detected by the sensor and can be used to trigger an alert or log the presence of debris, depending on the system's configuration.

The following examples are presented to illustrate non-limiting applications for use of the sensor devices of the present invention.

### In-Situ Flow-By Applications

In a flow-by application, the sensor device performs in-situ testing of the fluid. The sensor is positioned so that the sensing head is exposed to the fluid flow requiring measuring. First, the system under evaluation is analyzed by the end user to determine what locations requiring monitoring. Then the system is analyzed to determine what “chip load” is problematic for each of these monitoring locations. Then, the capture efficiency of the system is characterized. Faster fluid flows and weaker magnets will let some debris move past the sensor and remove less debris from the system. Using this information, the sensor device is designed to trip at a specific debris load. Then by monitoring the sensor, the system is evaluated throughout its operating life.

### In-Situ Reservoir Applications

In this embodiment the debris sensor device is incorporated into a reservoir. Incorporating the sensor into the reservoir can't narrow down which system components are in distress, but still indicates overall health of the system. Additionally, the capture efficiency of the sensor device is much higher because the magnet has time to slowly draw particles to it.

### Ex-Situ Testing

A fluid requiring examination could also be analyzed ex-situ after being removed from the system if the system does not have an oil filter. An example of this would be analyzing the oil from a gear motor, air compressor, lawn mower, manual transmissions, and differentials to check for debris after changing the oil on it.

For the avoidance of doubt, the devices and methods of the present invention encompass all possible combinations of the components, including various ranges of said components, disclosed herein. It is further noted that the term ‘comprising’ does not exclude the presence of other elements. However, it is also to be understood that a description of a product comprising certain components also discloses a product consisting of these components. Similarly, it is also to be understood that a description on a process comprising certain steps also discloses a process consisting of these steps.

In accordance with the patent statutes, the best mode and preferred embodiment have been set forth; the scope of the invention is not limited thereto, but rather by the scope of the attached claims.

What is claimed is:

1. A sensor device for detecting ferromagnetic metal debris in a fluid, comprising:
a housing having a sensor head unit adapted to be exposed to a fluid potentially containing ferromagnetic metal debris, wherein the housing comprises a non-ferromagnetic material,
a magnet disposed in the housing, wherein the magnet generates a first magnetic field,
a magnetic field sensor disposed in the housing, wherein the magnetic field sensor is positioned at a location where the first magnetic field is not able to be detected by the magnetic field sensor, and
wherein during use the magnet attracts ferromagnetic metal debris that becomes attached to the housing adjacent to the magnet whereby the first magnetic field shifts and becomes a second magnetic field, and wherein the second magnetic field is detected by the magnetic field sensor.

2. The sensor device according to claim 1, wherein the magnet and magnetic field sensor are disposed in the head unit.

3. The sensor device according to claim 1, wherein the magnetic field sensor is one or more of a Hall effect sensor, an anisotropic magneto resistive sensor, a giant anisotropic magnetic resistive sensor and an anisotropic tunneling magnetoresistive sensor.

4. The sensor device according to claim 1, wherein the head unit has a front end, and wherein the magnetic field sensor is located closer to the front end of the head unit as compared to the magnet.

5. The sensor device according to claim 1, wherein the magnet is one or more of a ring magnet, a plurality of one or more of rod and bar magnets arranged in the form of the ring, two rod magnets or two bar magnets.

6. The sensor device according to claim 1, wherein the sensor head unit is substantially cylindrical in shape.

7. The sensor device according to claim 6, wherein the magnetic field sensor is located closer to a radial center of the head unit as compared to the magnet.

8. The sensor device according to claim 1, wherein the housing includes a base connected to the sensor head unit, wherein a printed circuit board is located within the base and has an arm that extends upwardly into the sensor head unit, and wherein the magnetic field sensor is connected to the arm of the printed circuit board.

9. The sensor device according to claim 8, wherein wiring is connected to the circuit board for controlling the magnetic sensor, and wherein the wiring extends outwardly from the base.

10. The sensor according to claim 1, wherein a first pole of the magnet is disposed closer to a front end of the magnetic field sensor head unit as compared to a second opposite pole of the magnet.

11. The sensor device according to claim 2, wherein the magnetic field sensor is one or more of a Hall effect sensor, an anisotropic magneto resistive sensor, a giant anisotropic magnetic resistive sensor and an anisotropic tunneling magnetoresistive sensor, wherein the head unit has a front end, and wherein the magnetic field sensor is located closer to the front end of the head unit as compared to the magnet.

12. The sensor device according to claim 11, wherein the magnet is one or more of a ring magnet, a plurality of one or more of a rod and bar magnets arranged in the form of the ring, two rod magnets or two bar magnets.

13. The sensor device according to claim 12, wherein the sensor head unit is substantially cylindrical in shape, wherein the magnetic field sensor is located closer to a radial center of the head unit as compared to the magnet.

14. The sensor device according to claim 13, wherein the housing includes a base connected to the sensor head unit, wherein a printed circuit board is located within the base and has an arm that extends upwardly into the sensor head unit, and wherein the magnetic field sensor is connected to the arm of the printed circuit board.

15. The sensor device according to claim 14, wherein wiring is connected to the circuit board for controlling the magnetic sensor, and wherein the wiring extends outwardly from the base.

16. The sensor device according to claim 15, wherein a first pole of the magnet is disposed closer to a front end of the magnetic field sensor head unit as compared to a second opposite pole of the magnet.

17. A method for detecting ferromagnetic metal debris in a fluid, comprising the steps of:
obtaining the sensor device according to claim 1;
connecting the sensor device to an apparatus whereby the sensor head unit comes into contact with a fluid potentially containing ferromagnetic metal debris;
evaluating the fluid for ferromagnetic debris; and
sending an alert to an operator when a threshold debris level has been sensed.

18. The method according to claim 11, wherein the step of connecting the sensor device to the apparatus includes placing the sensor device in the apparatus at a location where fluid flows past the sensor.

19. The method according to claim 11, wherein the step of connecting the sensor device to the apparatus includes incorporating the sensor into a reservoir of the assembly.

20. A method for forming the sensor device according to claim 1, comprising the steps of:
obtaining the magnet that generates the first magnetic field;
determining a location where the first magnetic field is not able to be detected by the magnetic field sensor;
positioning the magnetic field sensor at the location; and
incorporating the magnet and magnetic field sensor in the housing.
